(19) United States
(12) Patent Application Publication
(10) Pub. No.: US 2017/0216466 A1
(43) Pub. Date: Aug. 3, 2017

(54) Ultraviolet-C Pocket Sterilizer Device and Method of Use

(71) Applicant: ONE HEALTH LABS, INC., Newberry, FL (US)

(72) Inventors: Mauricio Dujowich, Newberry, FL (US); Jennifer Jean Bentley, Newberry, FL (US)

(73) Assignee: ONE HEALTH LABS, INC., Newberry, FL (US)

(21) Appl. No.: 14/652,253

(22) PCT Filed: Feb. 4, 2015
(86) PCT No.: PCT/US15/14401

§ 371 Date: Jun. 15, 2015

Related U.S. Application Data
(60) Provisional application No. 62/068,046, filed on Oct. 24, 2014.

(51) Int. Cl. A61L 2/00; A61L 2/10

(52) U.S. Cl. CPC A61L 2/0047; A61L 2/10; A61L 2202/14; A61L 2202/16

(57) ABSTRACT

A sterilizer device adapted to deliver ultraviolet (UV) light to reduce the bacterial burden of skin incisions or wound surfaces in animals and humans. In one embodiment, the sterilizer device includes a microprocessor that is connected to a control unit, a user interface, an audio input/output, a connectivity module, a power source, a timer, a vibrating alert, a sensor for detecting a distance between a treatment area and the device, and a memory. The control unit is connected to a UV light source and an accelerometer. The user interface includes a display screen and control buttons. The audio input/output includes a speaker and a microphone. The connectivity module includes a USB port and/or a wireless connection. The sterilizer device is configured such that when a user activates one or more of the control buttons, the UV light source delivers a safe dose of UV-C that can kill bacteria.

### CROSS-REFERENCE TO RELATED APPLICATIONS

This application claims the benefit of U.S. Provisional Patent Application No. 62/068,046, filed Oct. 24, 2014, which is hereby incorporated by reference in its entirety.

### FIELD OF THE INVENTION

The present invention relates general to the field of medical devices. More particularly, the present invention is directed to a sterilizer device and the method of using the same, wherein the device has the unexpected ability to safely reduce bacterial burden of skin incisions or wound surfaces in animals and humans using ultraviolet (UV) light.

### BACKGROUND OF THE INVENTION

A surgical site infection (SSI) is an infection that occurs after surgery in the part of the body where the surgery took place. SSIs can be superficial infections involving the skin only. Other SSIs, however, are more serious and can involve tissues under the skin, organs, or implanted material. Such SSIs can lead to long-term disabilities or even death. Of many organisms that can cause SSIs, there is an alarming and increasing trend toward multi-drug resistant organisms such as the ESKAPE pathogens (Enterococcuss faecium, Staphylococcus aureus, Klebsiella pneumonia, Acinetobacter baumanii, Pseudomonas aeruginosa, and Enterobacter species).

Traditionally, SSIs are treated with antibiotics. However, increasing trend toward resistant organisms undermine the effectiveness of existing recommendations for antimicrobial prophylaxis. Additionally, antiseptics such as chlorhexidine, which are commonly used to disinfect surfaces prior to surgery, are also showing decreased effectiveness against bacteria. Thus, there is a need in the prior art for a new and improved disinfection method to reduce bacterial burdens and to prevent SSIs.

UVGI is a disinfection method that uses UV light at sufficiently short wavelengths to kill microorganisms such as bacteria. UVGI utilizes UV light in the “C” range, or “UV-C.” UV-C has a range of 100 to 280 nm, but a range of 200 to 280 nm is recognized as the most harmful to microorganisms. UV-C is effective in destroying the nucleic acids in these microorganisms so that their DNA is disrupted by the UV radiation, leaving them unable to perform vital cellular functions, such as reproducing. Without the ability to perform vital cellular functions, the microorganisms cannot cause or sustain an infection.

Germicidal UV may be delivered by a mercury-vapor lamp, a tunable laser, excilamp, UV-C LED, and microwave generated UV plasma that emits UV at the germicidal wavelength. Alternatively, one or more filters may be used to screen out wavelengths that are not desirable. Existing mercury-vapor lamps and other UV lamps are generally large apparatuses that are not designed to be portable, however. Accordingly, UVGI is not readily accessible, and is limited for use in a laboratory, a medical facility, or other sterile work facilities. Additionally, current methods of UVGI do not utilize devices that account for any change in conditions such as a distance between a treatment area and a UV light source and movement speed during use or offer automatic feedback to the user. Furthermore, a device that is optimized to deliver a safe dose of UV-C for the purposes of reducing bacterial burden of skin or wounds to prevent SSIs does not currently exist. Thus, there is a need in the prior art for a new and improved portable sterilizer device that can disinfect skin incisions or wound surfaces using UV-C.

### SUMMARY OF THE INVENTION

In view of the disadvantages inherent in the known types of apparatus that deliver germicidal UV now present in the prior art, the present invention provides a UV-C portable sterilizer device wherein the same can be utilized for disinfecting skin incisions or wound surfaces, thereby preventing or shortening the duration of infection on skin and muscles on the body of an animal or human.

In one embodiment, the UV-C portable sterilizer device in the present invention provides a compact housing member and a handle attached thereto, wherein the housing member comprises a defined interior volume. The interior volume of the housing comprises a microprocessor that is connected to a control unit, a user interface, an audio input/output, a connectivity module, a power source, a timer, a vibrating alert motor, a sensor for detecting a distance between a treatment area and the device, indicator lights, and a memory.

The control unit is connected to a UV light source and an accelerometer. The user interface includes a display screen and control buttons, wherein the display screen may be a touch screen. The audio input/output includes a speaker for emitting audible sounds and a microphone for inputting voice command. The connectivity module includes a USB port and/or a wireless connection such as a Bluetooth connection so that the device can connect to an external electronic device such as a computer to import data and export data recorded on the device. The sterilizer device is configured such that when a user activates one or more of the control buttons, the UV light source delivers a safe, consistent, and reliable dose of UV-C that can kill bacteria.

The present invention is advantageous in that it provides portable means for providing UVGI. Thus, the present invention can be readily used during travel and outside of primary care facilities, as well as in various types of sterile work facilities. Additionally, the present invention allows the user to adjust the dose of UV-C based upon various factors, including, but not limited to the treatment area, type of skin incisions or wound, and the distance between the treatment area and the device, among others. Accordingly, the present invention advances personalized medicine to create a more unified treatment approach specific to the individual needs, thereby providing a more efficient therapy.

It is therefore an object of the present invention to provide a UV-C portable sterilizer device that is dimensioned so as to allow one to carry the device upon one's person.

It is another object of the present invention to provide a UV-C portable sterilizer device that is configured to deliver a safe, reliable, and consistent dose of germicidal UV-C.

It is still another object of the present invention to provide a UV-C portable sterilizer device that can be controlled via voice command, control buttons, and/or touch screen.

It is still another object of the present invention to provide a UV-C portable sterilizer device that is configured to detect a distance between the surface of the treatment area and the device so as to more precisely deliver a safe and reliable dose of UV-C.

It is still another object of the present invention to provide a UV-C portable sterilizer device having an accelerometer to measure movement speed of the device.

It is still another object of the present invention to provide a UV-C portable sterilizer device that comprises connectivity means to communicate with external electronic devices to import and/or export data.

It is still another object of the present invention to provide a UV-C portable sterilizer device that can be sterilized by means such as ethylene oxide gas sterilization or gas plasma sterilization.

A final object of the present invention is to provide a UV-C portable sterilizer device that may be readily fabricated from materials that permit relative economy and that are commensurate with durability.

In the light of the foregoing, these and other objects are accomplished in accordance of the principles of the present invention, wherein the novelty of the present invention will become apparent from the following detailed description and appended claims.

### BRIEF DESCRIPTION OF THE DRAWINGS

The above and other objects and advantages of the present invention will be apparent upon consideration of the following detailed description, taken in conjunction with the accompanying exemplary drawings, in which like reference characters refer to like parts throughout, and in which:

FIG. 1 is a top perspective view of an exemplary embodiment of the present invention.

FIG. 2 is a bottom perspective view of an exemplary embodiment of the present invention.

FIG. 3 is a diagram of an exemplary embodiment of the present invention.

FIG. 4 is a flowchart showing steps for utilizing an exemplary embodiment of the present invention.

### DETAILED DESCRIPTION OF THE INVENTION

The present invention is directed towards a UV-C portable sterilizer device. For purposes of clarity, and not by way of limitation, illustrative views of the UV-C portable sterilizer device are described with references made to the above-identified figures. Various modifications obvious to one skilled in the art are deemed to be within the spirit and scope of the present invention.

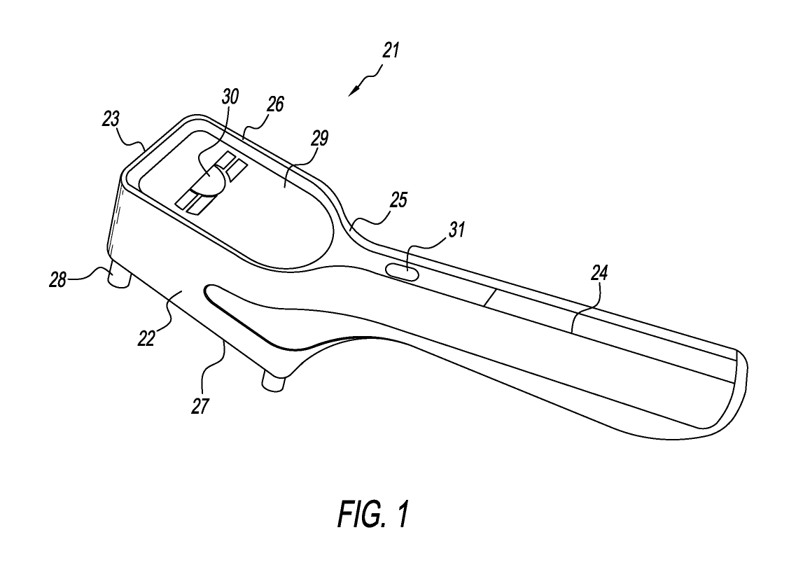

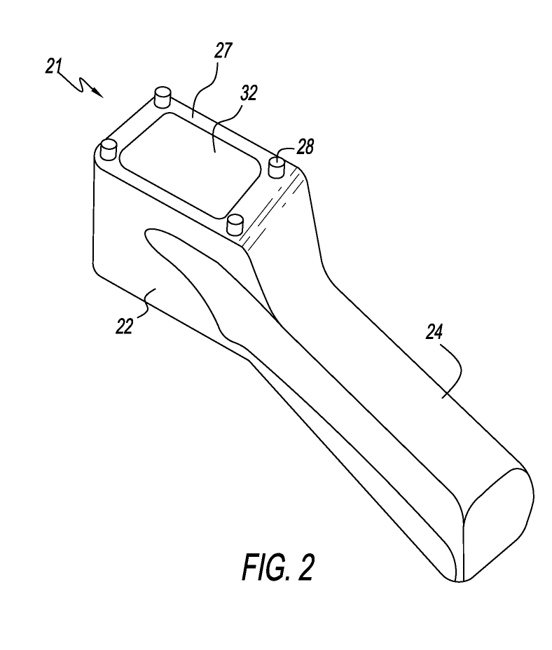

Referring now to FIGS. 1 and 2, there is shown a top perspective view and a bottom perspective view of an exemplary embodiment of the present invention, respectively. The UV-C portable sterilizer device 21 of the present invention comprises a housing member 22 having a proximal end 23 opposite a distal end 25, and an upper side 26 opposite a lower side 27. The housing member 22 further comprises a defined interior volume containing various electrical components embedded therein, wherein the electrical components are internally powered via batteries or other similar power source. Accordingly, it is contemplated that the housing member 22 or the handle 24 comprises a battery compartment or a power adaptor accessible from the exterior thereof. In some embodiments, the housing member 22 further comprises indicator means for notifying the user when the battery power is low. For example, the indicator means may comprise an indicator light that is activated when the battery power is low.

In the illustrated embodiment, the distal end 25 of the housing member 22 comprises an elongated handle 24 integral thereto. The handle 24 may be ergonomically shaped so as to provide comfort to the user when handling the device 21. It is contemplated that the housing member 22 and the handle 24 are completely sealed around the exterior thereof so as to prevent water or moisture from permeating therethrough. In other embodiments, it is contemplated that the housing member 22 does not comprise a handle so that the device is substantially pod-like in shape and structure.

The upper side 26 of the housing member 22 comprises a digital display screen 29 and control buttons 30, wherein the display screen 29 may be a touch screen. The display screen 29 comprises a liquid crystal display (LCD), although other types of displays, such as light emitting diode (LED) displays, can be used, depending upon embodiment. Without limitation, the display screen is adapted to display information such as the date, time, treatment mode, duration of use, status of the power source, and status of the device. For example, the status may include, “actively delivering treatment,” “idle,” or “warming up,” among others.

One or more of the control buttons 30 may be used to activate the device or the device 21 may comprise a separate power switch 31 as depicted in FIGS. 1 and 2. In the illustrated embodiment, the power switch 31 is disposed on the handle 24. The control buttons 30 and the power switch 31 typically include some combination of buttons, toggle switches, joysticks, or rotary dials. In some embodiments, the control buttons 30 are provided by using a touch screen overlay on the display screen 29 having one or more touch-sensitive user control elements. The control buttons 30 and the power switch 31 are operatively connected to the electrical components embedded within the interior volume of the housing member 22.

The lower side 27 of the housing member 22 comprises a UV light source 32 and a plurality of skin spacers 28 composed of plastic, dense rubber, or other suitable materials. The skin spacers 28 are elongated in shape and comprise a rounded tip so that the skin spacers 28 do not protrude into the skin. In one embodiment, the skins spacers 28 comprise at least 5 mm in length; the length of the skin spacers 28 depends upon embodiment, however. The skin spacers 28 are equal in dimension and shape so as to keep the device 21 level on the treatment area during use. The skin spacers 28 extend perpendicular from the lower side 27 of the housing member 22 to prevent the device 21 from directly contacting the treatment area. In the illustrated embodiment, each skin spacer 28 is located at the corners of the lower side 27 of the housing member 22 so that the skin spacers 28 are arranged in a rectangular configuration. In other embodiments, the skin spacers 28 may be arranged in various configurations.

The UV light source 32 comprises a lamp that is adapted to provide a peak emission between 200 nm and 280 nm. The UV light source 32 may be equipped with a UV-C bandpass filter, depending upon embodiment. The UV light source 32 can be activated and controlled via one or more control buttons 30 disposed on the upper side 26 of the housing member 22. Without limitation, the control buttons 30 may be used to adjust the wavelength of the light, the duration of exposure, and/or the frequency of the exposure, as a specific wavelength or a certain amount of exposure may be desirable for use on different treatment areas and/or type of skin incisions or wound, among other factors.

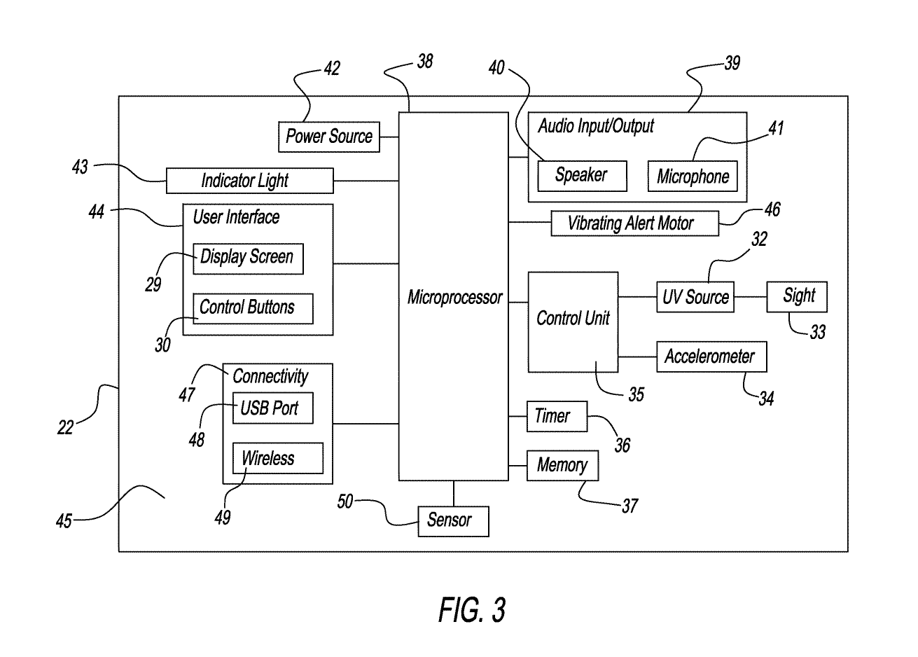

Referring now to FIG. 3, there is shown a diagram in accordance with an exemplary embodiment of the present invention. The housing member 22 comprises an interior volume 45 that is configured to embed electrical components therein. The electrical components include a microprocessor 38 that is connected to a control unit 35, a user interface 44, an audio input/output 39, a connectivity module 47, a power source 42 such as batteries, a timer 36, indicator lights 43, a vibrating alert motor 46, a sensor 50, and a memory 37.

The memory 37 may include a read only memory (ROM) or a programmable read only memory (PROM), which stores firmware instructions that are executed by the microprocessor 38. In some embodiments, the memory 37 may comprise a plurality of memory units. Additionally, the memory 37 is adapted to automatically store information such as the date, time, type of treatment, and treatment duration after each use, wherein such information can be recorded via the timer 36.

The user interface 44 includes the display screen 29 and the control buttons 30, wherein the display screen 29 and the control buttons 30 are operatively connected to the control unit 35 via the microprocessor 38. The microprocessor 38 produces menus and low-resolution color images that are stored in the memory 37 and that are displayed on the display screen 29. Additionally, the microprocessor 38 processes and executes commands input via the display screen 29 and the control buttons 30. When the user activates one or more of the control buttons 30, the microprocessor 38 communicates with the control unit 35 to actuate the UV light source 32 and deliver a safe dose of UV-C that can kill bacteria.

The user interface 44 may be used in conjunction with the indicator lights 43 on the exterior of the housing member 22. The indicator lights 43 are connected to the microprocessor 38 to deliver status information of various components of the device. Additionally, the indicator lights 43 are adapted to change colors, and can flash, and/or remain steady. Thus, one indicator light 43 can be used to deliver multiple messages. For example, one indicator light 43 may illuminate when the device is ready for use, change colors when the battery power is low, and flash when the UV light source 32 is delivering a dosage of UV-C.

The sensor 50 is adapted to detect a distance between the surface of the treatment area and the device. The sensor 50 may comprise an optical sensor, an ultrasonic sensor, a laser sensor, or the like. The specific type of the sensor, however, is not of primary relevance with regard to the intent of the present invention, which portends to provide a portable device that sterilizes skin incisions and wounds using UV-C. The sensor 50 detects how far the device is from the treatment area by measuring a straight line from a point on the surface of the treatment area to a point on the lower side of the device, preferably where the UV light source 32 is located, wherein the two points are directly aligned. The sensor 50 transmits the measured distance to the microprocessor 38. The microprocessor 38 utilizes various algorithms for determining the appropriate dose of UV-C based on the measured distance, and then relays the information to the control unit 35.

The control unit 35 is connected the UV light source 32 and an accelerometer 34. The UV light source 32 can be used in conjunction with a sight 33 to ensure that the UV light source 32 is properly aligned with the treatment area. It is contemplated that the sight 33 is adapted to find at least one reference point on the treatment area so that the UV-C will reach the treatment area when the user focuses on one of the reference points. The control unit 35 can regulate the wavelength and intensity of the light emitted from the UV light source 32 via the display screen 29 and/or the control buttons 30. For example, the desired wavelength and intensity may depend on the distance from the skin and the size of the treatment area. Additionally, the control unit 35 can control the duration and delivery method via the display screen 29 and/or the control buttons 30. For example, the control unit 35 can send instructions to the UV light source 32 to emit light at regular intervals, in pulses, or in a steady stream.

The accelerometer 34 guides the user to move the device across the treatment area at an appropriate speed. The microprocessor 38 calculates the target speed and the control unit 35 regulates the appropriate speed based on several factors such as treatment duration and the treatment area, among others. The microprocessor 38 is in electrical communication with alerting means such as the display screen 29, the speaker 40, and/or the vibrating alert motor 46 to indicate whether the device is moved too quickly or too slowly across the treatment area. In this way, the alerting means may be visual, auditory, or tactile. For instance, the present invention may be adapted to emit light on the display screen 29 or emit beeps through the speakers 40 to alert the user that the device is moved too quickly or too slowly across the treatment area. Additionally, the vibrating alert motor 46 is adapted to vibrate the device to alert the user that the device is moved above or below the target speed.

The audio input/output 39 includes a speaker 40 and a microphone 41. It is contemplated that the present invention includes an audio codec that receives an audio signal from the microphone 41 and provides an audio signal to the speaker 40. The microphone 41 and the speaker 40 can be controlled via the display screen 29 and/or one or more of the control buttons 30. The speaker 40 can be used to playback an audio track, a sound, or an alarm, such as beeps and chimes. Additionally, the speaker 40 can be used in conjunction with the user interface 44. For example, the speaker 40 can be used to provide various audible sounds when control buttons 30 are depressed, or that a particular selection has been made. The microphone 41, the audio codec, and the microprocessor 38 can be used to provide voice recognition, so that the user can input to the microprocessor 38 by using voice commands, rather than control buttons 30.

The microprocessor 38 also interfaces with the connectivity module 47, which includes a USB port 48, wherein the USB port 48 comprises a micro USB port, and/or a wireless connection 49 such as Bluetooth connection. The connectivity module 47 allows the device to be connected to an external electronic device such as a computer so that the stored data in the memory 37 can be uploaded to the computer to help document user compliance. Conversely, data, such as software updates can be downloaded from external electronic devices.

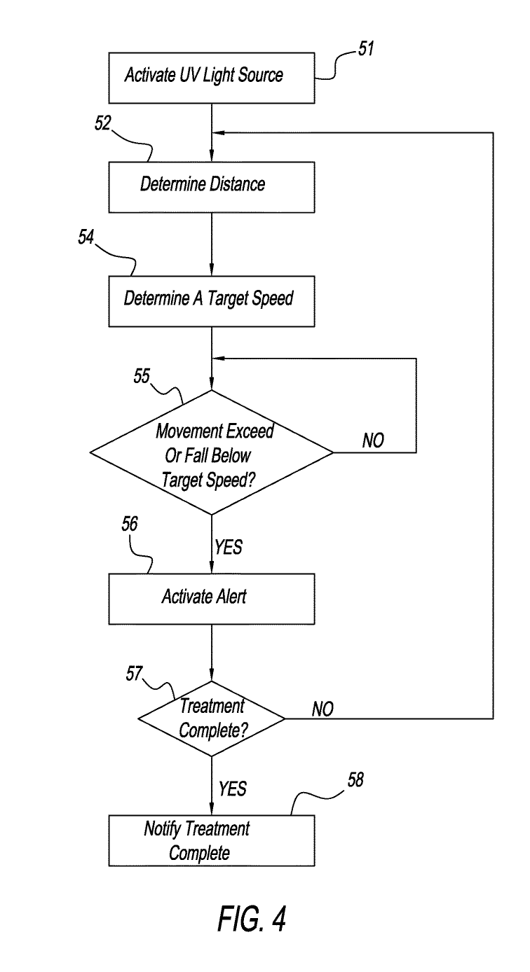

Referring now to FIG. 4, there is shown a flowchart showing steps for utilizing an exemplary embodiment of the present invention. In activate UV light source step 51, the UV light source is actuated and warmed until a stable output is achieved, if necessary. Thereafter, the sensor determines the distance between the surface of the treatment area and the device 52 by measuring a straight line from a point on the surface of the treatment area to a point on the lower side of the housing member. Preferably, the two points align such that the point on the lower side of the housing member is directly above or below the point on the surface of the treatment area. The sensor continuously monitors the distance because it can vary as the device is moved across the treatment area. Based on the measured distance, the microprocessor determines the dose of UV-C necessary to sterilize the treatment area. Alternatively, the dose of UV-C may be predefined so that the dosage of UV-C is not dependent upon the measured distance.

Thereafter, the microprocessor determines a target speed 54 in which the user must move the device across the treatment area. The target speed is defined as the speed most appropriate for ensuring that the UV light source is exposed to the treatment area for a sufficient amount of time. In some embodiments, the target speed may comprise a range. Without limitation, the target speed may vary depending on the type of incision or wound treated, the treatment area, or any combination thereof. In some embodiments, the target speed may be a predefined speed that allows for the treatment area to be sufficiently exposed to the UV light source.

The accelerometer continuously measures the speed in which the device is moved across the treatment area. If the device is moved across the treatment area above the target speed or if the speed variation is too great 55, the user is alerted 56 via an audible alarm, visual alarm, and/or a vibrating alert. In this way, the present invention ensures that the device is not moved too rapidly and that the treatment area is sufficiently exposed to the UV light source. Similarly, if the device is moved across the treatment area below the target speed or if the speed variation is too small 55, the audible alarm, visual alarm, and/or the vibrating alert is activated 56. In this way, the present invention ensures that the device is not moved too slowly and prevents the treatment area from being overly exposed to the UV light source.

It is contemplated that the device can emit beeps or other sounds through the speakers, and the backlight on the display screen or the indicator lights can illuminate so as to grab the user's attention. Additionally, the vibrating alert motor can vibrate the device until the movement speed matches the target speed. Thereafter, the device can send a status inquiry to determine whether the treatment has been completed 57. If the treatment is completed, the device is adapted to notify the user that the treatment is completed 58 via the display screen, the speaker, and/or the indicator lights. If the treatment is not completed, the device continues to operate. In some embodiments, the present invention can automatically deactivate the UV light source after a predefined period of time of non-activity to prevent the device from overheating, draining battery, or being left unattended.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1. A sterilizer device, comprising:
a housing member having an interior volume;
said interior volume comprising a microprocessor that is connected to a control unit, a user interface, a connectivity module, a power source, a memory;
said control unit connected to a UV light source that is visible from the exterior of said housing member;
said user interface comprising a display screen and control buttons;
wherein at least one of said control buttons is adapted to activate said UV light source to deliver a safe dose of UV-C.

2. The sterilizer device of claim 1, further comprising a sensor for detecting a distance between a treatment area and said housing member;
wherein said sensor is connected to said microprocessor.

3. The sterilizer device of claim 1, further comprising an accelerometer for measuring speed variations of said housing member;
wherein said accelerometer is connected to said control unit.

4. The sterilizer device of claim 1, said housing member further comprising a sight for aligning said UV light source with a treatment area.

5. The sterilizer device of claim 1, further comprising an audio input/output connected to said microprocessor, wherein said audio input/output having a speaker and a microphone.

6. The sterilizer device of claim 1, further comprising a connectivity module connected to said microprocessor.

7. The sterilizer device of claim 6, wherein said connectivity module comprises a USB port for connecting said memory to an external electronic device, thereby exporting data recorded on said memory to said external electronic device and importing new data to said memory from said external electronic device.

8. The sterilizer device of claim 6, wherein said connectivity module comprises a wireless connection for exporting data recorded on said memory to an external electronic device and importing new data to said memory from said external electronic device.

9. The sterilizer device of claim 1, further comprising a timer connected to said microprocessor.

10. The sterilizer device of claim 1, further comprising a vibrating alert motor connected to said microprocessor.

11. The sterilizer device of claim 1, further comprising a plurality of indicator lights connected to said microprocessor;
wherein one of said indicator light is adapted to indicate status of said power source.

12. The sterilizer device of claim 1, further comprising a handle attached to said housing member.

13. The sterilizer device of claim 1, wherein said display screen is a touch screen.

14. The sterilizer device of claim 1, further comprising a plurality of skin spacers extending outward from said housing member;
said plurality of skin spacers positioned around said UV light source.

15. A method of sterilizing a surgical site with a portable sterilization device, said portable sterilization device comprising:
a microprocessor that is connected to a control unit, a user interface, a power source, a memory;
said control unit connected to a UV light source;
said user interface comprising control buttons;
wherein at least one of said control buttons is adapted to activate said UV light source;
the method comprising the steps of:
activating said UV light source;
delivering a safe dose of UV-C via said UV light source to a treatment area.

16. The method of claim 15, said portable sterilization device further comprising:
a sensor connected to said microprocessor;
the method further comprising the steps of:
determining a distance between said treatment area and said portable sterilization device.

17. The method of claim 15, said portable sterilization device further comprising:
an accelerometer connected to said control unit;
the method further comprising the steps of:
determining a target speed;
measuring movement speed of said portable sterilization device across said treatment area.

18. The method of claim 17, said portable sterilization device further comprising:
an audio input/output connected to said microprocessor:
the method further comprising the steps of:
determining whether said movement speed exceeds or falls below said target speed;
activating alert if said movement speed exceeds or falls below said target speed.

19. The method of claim 17, said portable sterilization device further comprising:
a vibrating alert motor connected to said microprocessor:
the method further comprising the steps of:
determining whether said movement speed exceeds or falls below said target speed;
activating alert if said movement speed exceeds or falls below said target speed.

20. The method of sterilizing a surgical site of claim 15, further comprising the steps of:
determining whether treatment is complete;
notifying status information via said user interface.
